(19) United States
(12) Patent Application Publication
(10) Pub. No.: US 2009/0053809 A1
(43) Pub. Date: Feb. 26, 2009

(54) Retina-Specific Cells Differentiated In Vitro from Bone Marrow Stem Cells, the Production Thereof and Their Use

(76) Inventors: Axel R. Zander, Hamburg (DE); Katrin Engelmann, Dresden (DE); Monika Valtink, Dresden (DE); Claudia Lange, Hamburg (DE); Boris Fehse, Hamburg (DE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 11/719,377

(22) PCT Filed: Oct. 26, 2005
(86) PCT No.: PCT/EP2005/011468

§ 371 Date: May 9, 2008

(30) Foreign Application Priority Data
Nov. 16, 2004 (DE) 10 2004 055 615.6

(51) Int. Cl. C12N 5/06

(52) U.S. Cl. 435/377; 435/325

(57) ABSTRACT

The invention relates to the production of retina-specific cells from human adult bone marrow stem cells by culturing bone marrow stem cells in the presence of a differentiation medium. The invention also relates to retina-specific cells and to the use of these cells for treating diseases associated with acquired or congenital dysfunction of the retinal pigment epithelium, cells of adjacent structures of the entire retina and of the choroid coat as well as of other eye tissue.

The invention relates to retina-specific cells which are derived from human adult bone marrow stem cells, and to the production and use thereof for producing a pharmaceutical composition for the treatment of diseases associated with acquired or congenital dysfunction of the retinal pigment epithelium of the retina or of the choroid.

Degenerative disorders of the retina are one of the main causes leading to loss of sight. Such disorders frequently derive from disorders of the retinal pigment epithelium (RPE).

The cells of the RPE vary in size from 10 to 60 μm, with smaller cells in the fovea, which are highly pigmented owing to more and larger melanosomes, and larger and less strongly pigmented cells with few melanosomes on the peripheral retina. RPE cells are polarized with a villous apex on the apical side facing the photoreceptors and with a basal side with few folds. The apical side has microvilli which envelop the photoreceptor outer segments. The basal side faces Bruch's membrane on which the cells rest and to which they are “anchored”. RPE cells are moreover among the most metabolically active cells in the body and contain numerous mitochondria, rough endoplasmic reticulum, Golgi apparatus and a large round nucleus. A cell may occasionally contain 2 nuclei. The number of cells with two nuclei increases with age.

The role of RPE cells is diverse and includes various tasks
in vitamin A metabolism (e.g. on uptake of vitamin A from the bloodstream and conversion thereof into 11-cis-retinal, which is delivered to the photoreceptors, there binds to opsin and thus forms rhodopsin; the latter is deactivated by light during visualization, and is thus consumed and is transported in the consumed form back to the RPE where it is again converted into 11-cis-retinal);
as outer blood-retina barrier;
in the phagocytosis of outer segments of the photoreceptors, because each RPE cell is capable of uptake of up to 4000 discs of the outer segments of the photoreceptors per day, which are encapsulated in phagosomes and subsequently degraded in lysosomes;
in the absorption of light through absorption of the stray light, thus minimizing the latter;
in the formation of the interphotoreceptor matrix which is involved for example in the adhesion of the retina to the RPE;
in the active transport of water and metabolites such as, for example, D-glucose and tyrosine via Na⁺/K⁺-ATPase pumps on the apical surface and chloride-bicarbonate transporters on the basal surface;
in the response to mechanical and thermal damage through repair, regeneration, fibrovascular proliferation and pigment migration; and
in the trapping of toxins and free radicals through binding to the melanin located in the RPE, thus protecting the choroid and the retina as adjacent structures from oxidative damage.

Because of their predominant role in the eye, making vision possible, acquired or congenital dysfunctions of RPE cells, i.e. loss of cell integrity, proliferation or migration of the cells with the secondary consequence of degeneration of the non-regenerating photoreceptors, inevitably lead to subsequent irreversible loss of (central) vision.

In addition, the choriocapillary layer (lamina choroidocapillaris) basally adjacent to the RPE may, as a result of degeneration of the RPE, likewise degenerate, resulting in pathological neovascularization. This pathology is accompanied by bleeding from the new vessels and leads to a further deterioration in vision [HOLZ, F. G. & PAULEIKHOFF, D. (1996) Opthalmologe 93: 299-315].

Such a degeneration of the choroid frequently occurs during diabetes.

One example of an acquired retinal disorder having its cause in the RPE is the age-related macular degeneration (AMD), from which about 20% of patients over 65 suffer [WILLIAMS, R. A. et al. (1998) Arch Opthalmol 116: 514-520; YOUNG, R. W. (1987) Surv Opthalmol 31: 291-306]. Macular degeneration is the inexact historical term for a group of diseases which lead to dysfunctions or losses of function in the light-sensing cells in the macular area of the retina and eventually lead in a weakening loss of the vital central or peripheral vision. It has not to date been possible to elucidate adequately the pathogenesis of AMD [HOGAN, M. J. (1972) Trans Am Acad Opthalmol 0 to 1 76: 64-80; YOUNG, R. W. (1987) Surv Opthalmol 31: 291-306; LAHIRI-MUNIR, D. (1995) “Retinal Pigment Epithelial Transformation.” Springer-Verlag, Heidelberg].

One example of a congenital degeneration of the retina is retinopathia pigmentosa which comprises a group of disorders which are also referred to as retinitis pigmentosa and are characterized by degeneration of the retinal epithelium without accompanying inflammation, by atrophy of the optic nerve and extensive pigment alterations in the retina, which lead to a progressive decline in vision. Retinitis pigmentosa with its numerous subtypes is one of the commonest reasons for blindness particularly in people over the age of 30 [cf. LORENZ, B. et al. (2001) Dt. Arztebl 98: A3445-3451; Information of the Patients' Association “Pro Retina e.V.” under www.pro-retina.de].

Therapeutic approaches to the treatment or cure of retinal disorders in use at present, including laser therapy or surgical removal of neovascularization membranes, are initiated relatively late in the progress of the disease and are at best able only to retard the disease. There is at present no cure for retinal disorders.

The use of fully functional donor cells (RPE) as transplant to replace diseased cells provides a promising approach in the direction of curing such diseases. Donor cells are usually removed from donor eyes post mortum and are used either fresh or after a culturing step. Disadvantages of cells removed post mortum are a reduced vitality and, through the culturing step, an impaired differentiation status of the cells. Despite these disadvantages, it has been possible to achieve transplants with medium-term success in animal models [cf. ALGERVE, P. V. et al. (1997) Graefe's Arch Clin Opthalmol 235: 149-158; CRAFOORD, S. et al. (1999) Acta Opthalmol Scand 77: 247-254; GOURAS, P. et al. (1985) Curr Eye Res 4: 253-265; GOURAS, P. et al. (1989) Prog Clin Biol Res 314: 659-671; LI, L. et al. (1988) Exp Eye Res 47: 771-785; LI, L. et al. (1991) Exp Eye Res 52: 669-679; LITTLE, C. W. et al. (1996) Invest opthalmol V is Sci 37: 204-211; PEYMAN, G. A. et al. (1991) Ophthal Surg 22: 102-108; SHEEDLO, H. J. et al. (1989) Exp Eye Res. 48: 841-854; SEILER, M. J. & ARAMANT, R. B. (1998) Invest Opthalmol V is Sci 39: 2121-2131]. However, attempts at transplantation in human patients have failed owing to the poor quality of the donor cells. Other retinal cells such as, for example, photoreceptors have to date been transplanted only experimentally and only as embryonic cell [cf. ARAMANT, R. B. et al. (1999) Invest Opthalmol V is Sci 40: 1557-1564], and thus this approach is at present unacceptable for therapy according to current scientific and ethical standards.

In view of unsolved problems, the object on which the invention is based is to provide a therapy for retinal pathologies.

In accordance with the present invention, this problem is solved by differentiating mesenchymal or hematopoietic stem cells from bone marrow or a mixture of both cell types into retina-specific cells, especially by methods as claimed in claims 1 to 29 and 43, a use as claimed in any of claims 30 to 38 and 50 to 53, cells and cell preparations as claimed in claims 39 to 42 and 44 to 47, respectively, and/or a pharmaceutical preparation as claimed in claim 49.

### DESCRIPTION OF THE FIGURES

FIG. 1: Light micrograph of isolated, undifferentiated mesenchymal stem cells after 2 days in culture in the presence of CCM as differentiating medium (magnification ×100).

FIG. 2: Light micrograph of isolated mesenchymal stem cells after 5 days in culture in the presence of CCM as differentiating medium with insipient differentiation of the stem cells into cells with an apparently neural cell morphology which are characterized by the formation of dendritic processes (magnification ×100).

FIG. 3: Light micrograph of isolated mesenchymal stem cells after 9 days in culture in the presence of CCM as differentiating medium with a further advance in the differentiation of the stem cells into cells with an apparently neural cell morphology, which are characterized by insipient branching of the dendritic processes (magnification in FIG. 3A×100 and FIG. 3B×200).

FIG. 4: Light micrograph of isolated mesenchymal stem cells which, after culture in the presence of CCM as differentiating medium for 14 days, show a neural cell morphology with dendritic processes and branches (magnification in FIG. 4A×100, FIG. 4B×200 and FIG. 4C×320).

FIG. 5: Chromatogram (violet curve—214 nm; blue curve—280 nm) of fractions 20-50 of a culture medium before incubation of choroid in this medium (upper part of the figure) and of a conditioned medium which is obtained after incubation with choroid in this medium (lower part of the figure); representation of the changes in the protein composition.

The term “retina-specific cells as used herein refers to a subgroup of neural cells which occur naturally in the retina. This term additionally includes cells having neural morphology which resemble specific cells from the retina and carry out their function(s).

The term “stem cells” as used herein refers to adult mesenchymal or hematopoietic stem cells from the bone marrow which can be obtained from a bone marrow aspirate by suitable methods known to the skilled worker. These methods for obtaining bone marrow are harmless for the donor and are carried out during a minor operation.

In a preferred embodiment of the invention, isolated and expanded stem cells from bone marrow are differentiated into retina-specific cells using a method in which
a) the stem cells are expanded in a suitable culture medium;
b) the expanded stem cells are cultured in a differentiating medium; and
c) the retina-specific cells are isolated by separating the cells from the differentiating medium.

In one embodiment of the invention, adult mesenchymal stem cells from bone marrow are used as starting material in this differentiation method.

It has surprisingly been possible to show that the method of the invention leads to a differentiation into retina-specific cells.

It has moreover been possible to show for the first time ever that mesenchymal stem cells with their known ability to differentiate into a large number of different cells such as, for example, bone, cartilage, lung, spleen, central nervous system, muscles and liver cells (cf. PEREIRA, R. F. et al. (1995) Proc Natl Acad Sci USA 92: 4857-4861; AZIZI, S. et al. (1998) Proc Natl Acad Sci USA 95: 3908; FERRARI, G. et al. (1998) Science 279: 1528-1530; KOPEN, G. C. et al. (1999) Proc Natl Acad Sci USA 96: 10711-10716) can also be differentiated in vitro into retina-specific cells.

The mesenchymal stem cells used according to the invention express at least two typical surface antigens selected from the group consisting of CD59, CD90, CD105 and MHC I. The mesenchymal stem cells of the invention are, however, characterized not solely by the expression of one or more specific surface markers, but generally by the expression pattern of a large number of antigens which is distinguished by the detectability (expression present) or lack of detectability (no expression present) of these antigens in specific detection methods. Thus, for example, no expression of CD34 and CD45 is measurable. In a particularly preferred embodiment, the mesenchymal stem cells express the surface antigens CD105 (endoglin) and CD90 (Thy-1).

The expression of these specific markers (surface antigens) can be detected by commercially available antibodies having specificity for the respective antigens, using standard immunodetection methods [cf. LOTTSPEICH F. & ZORBAS H. “Bioanalytik”, Spektrum Akademischer Verlag GmbH, Heidelberg-Berlin (1998)}]. For example, the complete MHC I complex is detected using the antibody against HLA-A,B,C (manufacturer BD Pharmingen, catalog number 555552).

During the proliferation or growth phase of the cells, a varying number of cells adheres to the base or to the wall of the respective culture vessel. The adherently growing, expanded mesenchymal stem cells are used for differentiation into the retina-specific cells in stage b) of the method of the invention (cf. Example 2).

In a further embodiment of the invention, adult hematopoietic stem cells from bone marrow are used as starting material in the differentiation method of the invention.

The hematopoietic stem cells used according to the invention express at least one typical surface antigen selected from the group consisting of CD34 and CD45. The hematopoietic stem cells of the invention are, in analogy to the mesenchymal stem cells of the invention, likewise characterized not solely by the expression of one or more specific surface markers, but generally by the expression pattern of a large number of antigens. In a particularly preferred embodiment, the hematopoietic stem cells express the surface antigens CD34 and CD45.

Expression of the specific markers (surface antigens) for the hematopoietic stem cells can likewise be detected by commercially available specific antibodies through use of standard immunodetection methods [cf. LOTTSPEICH F. & ZORBAS H. “Bioanalytik”, Spektrum Akademischer Verlag GmbH, Heidelberg-Berlin (1998))]

The hematopoietic stem cells of the invention can be purified by means of MACS (“magnetic-activated cell sorting”; from Miltenyi). Purification by this technique takes place on columns which are situated inside a magnet and onto which are put the bone marrow cells which have been incubated with antibodies which are coupled to ferromagnets. Complexes of stem cells and antibodies bind to the column and can thus be specifically purified [SUTHERLAND, et al. (1996) J Hematotherapy 5: 213-226]. Further methods are familiar to the skilled worker.

The hematopoietic stem cells are preferably used immediately after their purification for the differentiation into retina-specific cells in stage b) of the method of the invention. However, the invention also includes further culture or expansion of the purified cells.

In a further embodiment of the invention in turn, stem cells from bone marrow which include both mesenchymal and hematopoietic stem cells are used as starting material in the differentiation method of the invention. Included therein according to the invention is both direct use of aspirate taken from bone marrow, and any mixture which comprises the previously isolated mesenchymal and the previously isolated hematopoietic stem cells subsequently reunited.

After the retina-specific cells have been obtained in stage c) of the differentiation method, they are preferably suspended in a suitable cell culture medium and then deep-frozen for storage without loss of their therapeutic potential. This medium is preferably a standard medium selected from the group consisting of RPMI, medium 199, DMEM (low glucose; this medium corresponds to modified Eagle's medium (Gibco 31885) and Iscove's medium, in each case alone or as 1:1 mixture with Ham's F12 nutrient mixture. The medium may further be a special medium selected from the group consisting of human endothelial SFM medium (Gibco 11111), START V (Biochrom F8075) and Neurobasal or Neurobasal-A medium (Gibco 21103 or 10888) and their supplements N-2 (Gibco 17502) or B27 (Gibco 17504-044). These media are employed with or without addition. A possible addition for said special media is Ham's F12 nutrient mixture which has a high content of amino acids and vitamins. DMSO or methylcellulose as cryoprotectant, and proteins to stabilize sensitive biological substances, are preferably added to the selected medium.

10% DMSO as cryoprotectant and at least 10% serum (or albumin in the case of serum-free culture) to stabilize sensitive biological substances are particularly preferably added.

DMEM medium (low glucose) can optionally be used with HEPES (Gibco 22320) as additional buffer substance or without this addition. HEPES as buffer substance stabilizes the pH of the medium more efficiently than for example a carbonate or phosphate buffer and is well tolerated by the stem cells.

It should be noted in relation to the use according to the invention of Neurobasal or Neurobasal-A medium that these media are preferably used only to culture the differentiated cells obtained in stage c) of the method of the invention, because the viability of undifferentiated stem cells is drastically reduced in these media.

The in vitro differentiation of the retina-specific cells of the invention, and the initiation of differentiation of the cells (the “priming”), which is not morphologically visible and is completed only after transplantation of the cells into the eye under the influence of the surrounding tissue, takes place in a simple and reliable manner by culturing the cells in step b) in a special medium. This medium comprises either the supernatant of a culture medium in which choroids and/or parts thereof have been cultured (cf. Example 3), or the supernatant obtained after complete homogenization of retina by centrifugation (see Example 4). This medium is referred to hereinafter generally as “differentiating medium”.

The differentiating medium preferably comprises choroid-conditioned medium (CCM) or retina extract (RE) [cf. PFEFFER, B. A. (1991) Prog Retina Res 10: 251-291; HO, J. & BOK, D. (2001) Mol V is 7: 14-19; VENTURA, A. C. et al. (1996) Opthalmologie 93: 262-267; VALTINK, M. et al. (1999) Graefe's Arch Clin Exp Opthalmol 237: 1001-1006; COULOMBE, J. N. et al. (1993) Neuron 10: 899-906].

In a particular embodiment, CCM can also be employed in conjunction with RE.

A method which can be used to obtain the choroid-conditioned medium for differentiating the stem cells into retina-specific cells is one in which
a) the anerior segment, the vitreous and the neurosensory retina are removed from human donor eyes;
b) the choroid and/or fragments thereof are dissected out of the eye;
c) adherent cells belonging to the retinal pigment epithelium are removed from the dissected choroid and/or the fragments thereof by washing and subsequent incubation in the collagenase solution;
d) the choroid and/or fragments thereof is incubated in a suitable culture medium; and
e) the supernatant of the culture medium is collected after incubation has taken place as differentiating medium.

Standard cell culture media can be used as culture medium in step c) of this method (cf. examples). The choroid culture takes place at 37° C. in a moist atmosphere (90 to 97% humidity) in an incubator in a gas mixture composed of 5% CO₂ and 95% air.

In preferred embodiments of the invention, the culture media used to produce the choroid-conditioned medium are standard media such as RPMI, medium 199, DMEM (low glucose; corresponds to modified Eagle's medium (Gibco 31885)) or Iscove's medium, in each case alone or mixed 1:1 with Ham's F12 nutrient mixture. DMEM (low glucose) can optionally be used with HEPES (Gibco 22320) as additional buffer substance or without this addition. The culture medium also comprises fetal calf serum (FCS) as further addition.

A 1:1 mixture of medium 199 and Ham's F12 which is supplemented with 1% (v/v) FCS is preferably used for producing the choroid-conditioned medium.

A further possibility is to replace the serum in the medium for producing the choroid-conditioned medium by serum substitutes. These serum substitutes are preferably selected from the group consisting of insulin, albumin (Gibco 11020 or 11021), transferrin, selenium and further trace elements, lipids, lipoproteins, ethanolamine/phosphoethanolamine and further hormones such as hydrocortisone.

The serum substitutes insulin, transferrin and selenium are preferably employed according to the invention as ITS supplement (Gibco 51300). On use of the individual substances, the preferred concentration range of the individual substances is 1-10 μg/ml in the case of insulin, 1-20 μg/ml in the case of transferrin and 20 nM in the case of selenium.

The trace elements are preferably selected from the group consisting of manganese, tin, nickel, vanadium or molybdenum. Lipids and lipoproteins are preferably employed as prepared supplement optimized for the cell culture sector (e.g. Sigma F7175, L0288, L9655 or L0163).

Ethanolamine or phosphoethanolamine are added to the medium because they are essentially required by the cells both to assist lipid transport and in serum-free media or media without serum supplementation in phospholipid biosynthesis to construct the cell membrane. They are employed in the standard concentration usual for cell cultures of up to 50 μmol/l [cf. GRAFF, L. et al. (2002) Am J Pathol 160: 1561-1565; KIM, E. J. (1999) In Vitro Cell Dev Biol Anim 35(4): 178-182). Hydrocortisone is preferably employed in a concentration of 1-10 nM and serves to feed inter alia neural cells in the culture medium.

In a further preferred embodiment of the invention, the medium used in step c) to produce the choroid-conditioned medium by culturing choroid and/or fragments thereof is a synthetic serum substitute which comprises all the minimally necessary substances in prepared concentration. The synthetic serum substitute Biochrom K3611 or K3620 is particularly preferably used in this connection.

In one embodiment of the invention, the choroids are incubated over a period of from 2 to 8 days, preferably of 4 days, to produce the choroid-conditioned medium (CCM).

The supernatant of this culture is obtained as conditioned medium preferably at the end of the incubation. No incubation with interim removal of the supernatant to generate a larger amount of conditioned medium by combining the individual supernatants is carried out because this multiple “milking” of the culture leads to a differentiating medium of poorer quality and, in some cases, inhibiting effect.

In a preferred embodiment of the invention, the choroid of a donor eye are incubated, after enzymatic detachment of the cells belonging to the retinal pigment epithelium, in F99 to which 1% (v/v) FCS have been added for 4 days. After completion of the culture, the conditioned medium is obtained by centrifugation (cf. examples).

A method which can be used to obtain the retina extract (RE) for differentiating the stem cells into retina-specific cells (see Example 4) is one in which
a) the retina is isolated from human donor eyes;
b) the retina is homogenized with addition of proteinase inhibitors; and
c) the supernatant is obtained as retina extract from the homogenate by centrifugation.

The choroid-conditioned medium (CCM) and the retina extract (RE) as addition to the differentiating medium are in each case filtered under sterile conditions and stored at about −20° C. or employed directly for differentiating the mesenchymal stem cells (see examples). As shown above, the differentiation according to the invention of the stem cells into retina-specific cells, and the induction of this differentiation takes place by growing the stem cells in the presence of a differentiating medium which comprises either the supernatant of a culture medium in which choroids and/or parts thereof have been cultured, or the supernatant obtained after completion of homogenization of retina by centrifugation (cf. Examples 3 and 4).

In one embodiment of the invention, the stem cells are incubated in the presence of from 1 to 20% CCM and/or in the presence of from 0.1 to 5.0% RE.

In a preferred embodiment of the invention, the stem cells are incubated in the presence of 1-15% CCM and/or in the presence of 0.5-5% RE.

In a particularly preferred embodiment, the stem cells are incubated in the presence of 10% CCM and/or in the presence of 1% RE.

In one embodiment of the invention, the stem cells are cultured in the presence of the differentiating medium for a period of from 3 to 21 days for differentiation into retina-specific cells.

The stem cells are preferably cultured in the presence of the differentiating medium for a period of from 14 to 21 days for differentiation into retina-specific cells.

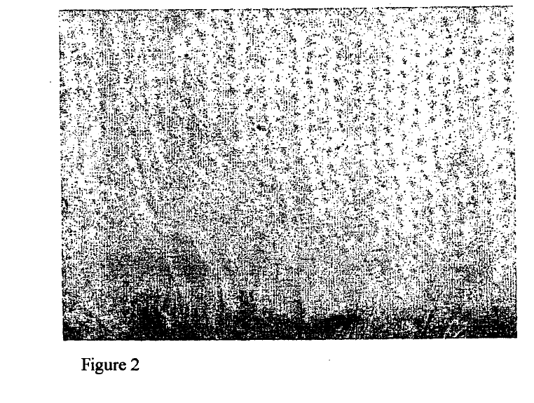

After 3 to 5 days, the first morphological changes due to the differentiation in the presence of the differentiating medium appear in the cells, which initially assume a stellar morphology. This change becomes manifest over the course of up to 3 weeks (cf. FIGS. 2 to 4). The cells are completely differentiated after 14 days in culture, because no further differentiation is to be observed, and the cells retain their changed, now apparently neuronal morphology.

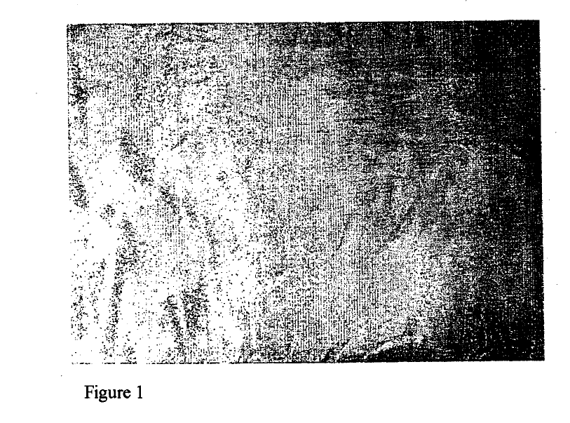

After the differentiation step, the cells differ from undifferentiated or completely differentiated cells through their morphology (cf. FIGS. 1 to 4). Undifferentiated cells are elongate (see FIG. 1), whereas cells after induction of differentiation into retina-specific cells form offshoots and assume a star-shaped, stellar form (see FIG. 2). Some of these star-shaped cells show granular collections with a dark appearance around the nucleus. The cells change their morphology as differentiation progresses further and form dendrite-like processes and branches. After about 9 days, the first apparently neural cells can be observed, while the stellar cells slowly became no longer detectable in the culture.

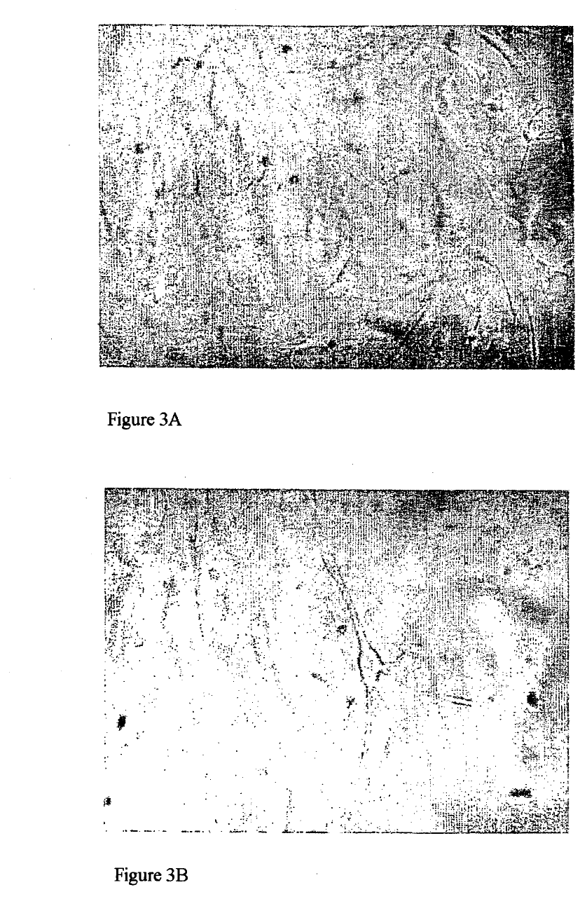

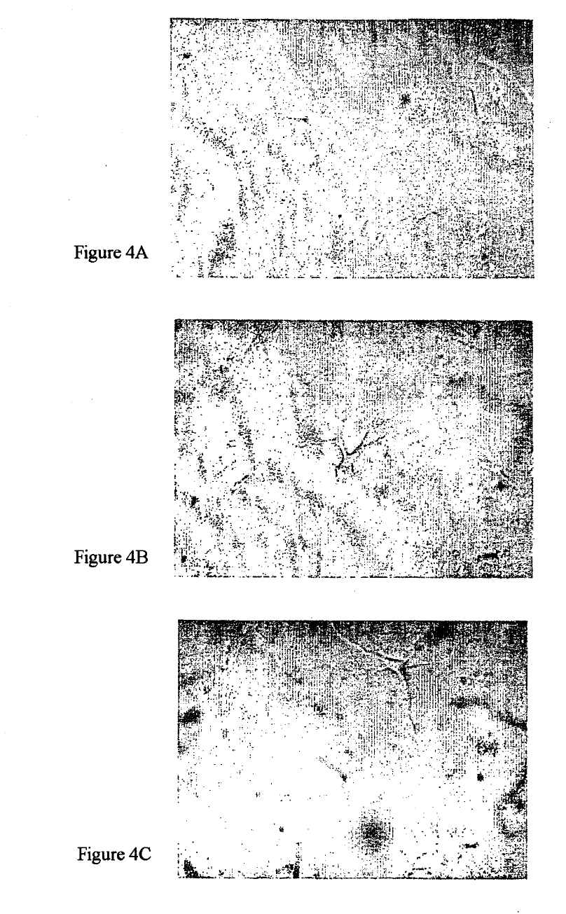

The apparently neural cells exhibit phenotypical similarity to astrocytes and oligodendrocytes which have been differentiated from cultured neural stem cells. After about 9 to 14 days, small swellings with the morphological appearance of podia appear on the ends of the branched cell offshoots (see FIGS. 3 and 4). As development continues, the cell offshoots which have already formed become thicker and scarcely any new cell offshoots are formed. This phenotype is maintained for the following 5 to 7 days.

In a further embodiment, the stem cells are cultured in the presence of the differentiating medium for only a short period of from 3 to 14 days in order to induce differentiation of the stem cells into retina-specific cells, in which case the differentiation process following the induction is not completed.

It is particularly preferred according to the invention for the stem cells to be cultured for initial differentiation into retina-specific cells for a period of from 3 to 9 days in the presence of the differentiating medium.

In these exemplary embodiments, completion of the differentiation of the initially differentiated or predifferentiated cells into retina-specific cells takes place after administration of the cells into the eye in vivo under the influence of the microenvironment of the eye.

In further embodiments, the stem cells are differentiated in a multistage method in which both CCM and RE are employed for differentiation. In this case, an initial differentiation, lasting 3 to 14 days, of the isolated and expanded stem cells in a CCM-containing differentiating medium is followed by culturing the cells in an RE-containing differentiating medium for up to 4 weeks.

In a particular embodiment of the invention, culturing in a CCM-containing differentiating medium for 3 to 5 days is followed by culturing the cells in an RE-containing differentiating medium for 1 to 14 days in order to obtain for example retinal pigment epithelium.

In a further particular embodiment, the stem cells are, after culturing in a CCM-containing differentiating medium, further differentiated in a special medium proven for neuron cultures, instead of in an RE-containing differentiating medium, in order to obtain neuronal cells.

This special medium used for further differentiation is preferably Neurobasal or START V.

Multistage culturing is also preferred according to the invention, where culturing in CCM-containing differentiating medium is followed by culturing for up to 2 weeks in a special medium proven for neuron cultures, such as, for example, Neurobasal or START V, in order to obtain retinal cells.

It is preferred according to the invention for the density of the stem cells during the incubation for differentiation into retina-specific cells in step b) of the method of the invention to be between 0.5×10³ and 2.5×10³ cells per cm², particularly preferably 2×10³ cells per cm².

Adjustment of the cell density is crucial for the differentiation of the stem cells into retina-specific cells and the change in the morphology of the stem cells to that of the target cells. The total number of stem cells which can be employed for the differentiation depends on the size of the culture vessel which defines the area on which the cells can grow. A total cell count in the range from 1×10³ to 2.5×10³ cells results on use of 24-well culture dishes with an area of 1.88 cm² per well available for growth when 2×10² to 5×10³ cells are seeded per well. It is particularly preferred to employ a total cell count at the lower end of the preferred range, because on plating out the cells are then present singly in the dish and proliferate slowly. Use of higher seeding densities of more than 5×10³ cells per well results in subconfluent to confluent cell cultures with strongly proliferating cells which, however, do not differentiate, and thus no changes in morphology occur.

The choroid-conditioned medium and the retina extract comprise in accordance with their use as addition to the differentiating medium of the invention one or more growth factors or subtypes thereof. The retina extract is additionally a supplier of further retinal trophic factors and additionally supplements the differentiating medium with lipoproteins and proteins, and vitamin A and vitamin A derivatives.

The potential risk, associated with the addition of biological supplements such as CCM, RE or FCS to the differentiating medium, of contamination of the differentiating medium with pathogenic organisms from the supplements can be avoided by substitution for these complex additions. The substitution takes place in this case by adding defined single substances which are present in the complex media and are selected from the group consisting of members of the FGF family (FGF: “fibroblast growth factor”), members of the NT family (NT: “neurotrophin”), members of the BMP family (BMP: “bone morphogenic protein”), PDGF (“platelet-derived growth factor”), EGF (“epidermal growth factor”), BDNF (“brain-derived neurotrophic factor”), CNTF (“ciliary neurotrophic factor”), HGF (“hepatocyte growth factor”) and NGF (“nerve growth factor”).

The naming of the individual growth factors includes according to the invention also their subtypes, whose use according to the invention is likewise claimed. The subtypes of growth factors are known to the skilled worker and include inter alia PDGF-AA, PDGF-BB and PDGF-AB.

In a preferred embodiment of the invention, the member of the FGF family is preferably basic fibroblast growth factor bFGF (FGF-2).

The members of the neurotrophin family are preferably NT-3 and NT-4.

The member of the BMP family is preferably BMP-4.

The effects of the growth factors BDNF, CNTF and growth factors of the NT family on the growth and survival of nerves and/or glial cells (BDNF) and the differentiation of various neuronal cell types (CNTF) are very diverse.

Whereas NT-3 on the one hand acts generally as a mitogen on retinal progenitor cells and thus promotes the formation of an undifferentiated cell pool from which all retinal cell types can be formed (DAS, et al. (2000) J Neurosci 20(8): 2887-2895), on the other hand it is also involved in neuronal development and promotes, with synergistic enhancement by BDNF, for example the outgrowth of neurites from neuronal precursor cells [HOSSAIN, et al. (2000) Exp Neurol 175(1): 138-151]. It has additionally been possible to demonstrate for NT-3 a cell cycle-controlling function in the progenitor cells of sensory neurones, the absence of which leads to cell cycle-dependent cell death [ELSHAMY, et al. (1998) Neuron 21(5): 1003-1015). A culture of neural progenitor cells from the embryonic striatum differentiates under the influence of neurotrophic factors such as NT-3 and CNTF into bipolar neurons and oligodendrocytes, whereas BDNF promotes differentiation into multipolar neurons [LACHYANKAR, et al. (1997) Exp Neurol 144(2): 350-360).

Neurotrophins such as NT-3, NT-4/5 and BDNF have an activity as “survival factor” for neurons of the striatum, because they are able to protect such cells from dying during degenerative disorders [PEREZ-NAVARRO, et al. (2000) J Neurochem 75(5): 2190-2199]. BDNF in combination with CNTF promotes the growth and branching of axons following lesions [LOH, et al. (2001) Exp Neurol 170(1): 72-84], while BDNF on its own is able to promote the differentiation of neuronal stem cells out of the hippocampus [SUZUKI, et al. (2003) Biochem Biophys Res Commun 309(4): 843-847).

The growth factors of the FGF family and of the BMP family, and HGF cooperate in the cell division of retinal cells. These cells include retinal gangliocytes (neurons), amacrine, bipolar and horizontal cells, photoreceptor cells (rods and cones), Müller's radial cells and retinal pigment epithelium (RPE). BMP-4 and BMP-7 as members of the BMP family are crucially involved in the development of various structures in the eye, such as retina, retinal pigment epithelium, ciliary pigment epithelium and optic nerve, which differentiate out of the neuroepithelium, and in making the neural connection between brain and retina at the optic disk [LIU, et al. (2003) Dev Biol 256(1): 34-48; ADLER, et al. (2002) Development 129(13): 3161-3171]. BMP-4 exerts its controlling action through promoting cell division and through targeted induction of programmed cell deaths [TROUSSE, et al. (2001) J Neurosci 15; 21(4): 1292-1301] and is able both to activate various signal transduction pathways in cells and to bring about the differentiation of stem cells into smooth muscle cells or into glia cells [RAJAN, et al. (2003) J Cell Biol 161(5): 911-921]. BMPs in general are crucially involved in the differentiation of cortical stem cells into neurons and astrocytes [CHANG, et al. (2003) Mol Cell Neurosci 23(3): 414-416], while BMP-7 is responsible for the development of the ciliary body of the eye [ZHAO, et al. (2002) Development 129(19): 4435-4442].

bFGF acts, depending on the concentration, both on endothelial cells of the cornea and on the retinal pigment epithelium either as mitogen or as differentiation factor. bFGF is further known to act as factor for retinal cells, especially for photoreceptor cells, which is able to ensure the survival of these cells [cf. GU, et al. (1996) Invest Opthalmol V is Sci 37: 2326-2334; ITAYA, et al. (2001) Am J Opthalmol 132: 94-100; TRAVERSO, et al. (2003) Invest Opthalmol V is Sci 44: 4550-4558; VALTER, et al. (2002) Growth Factors 20: 177-188; EZEONU, et al. (2000) DNA Cell Biol 19: 527-537; AKIMOTO, et al. (1999) Invest Opthalmol V is Sci 40: 273-279; STERNFELD, et al. (1989) Curr Eye Res 8: 1029-1037; SCHWEGLER, et al. (1997) Mol V is 3: 10].

Hepatocyte growth factor (HGF) stimulates the migration and proliferation of retinal pigment epithelium in vitro and thus promotes wound healing of RPE defects, with the newly formed cells assuming under the influence of HGF a distinctly epithelial morphology and becoming freely movable through loss of tight junctions (MIURA, et al. (2003) Jpn J Opthalmol 47: 268-275; JIN, et al. (2002) Invest Opthalmol V is Sci 43: 2782-2790]. HGF is also a growth and differentiation factor for neuronal stem cells and promotes the proliferation of neurospheres (cell aggregates consisting of neural progenitor cells) and the differentiation of neural stem cells into neurons [KOKUZAWA, et al. (2003) Mol Cell Neurosci 24: 190-197).

The invention further relates to the use of choroid-conditioned medium (CCM) for differentiating stem cells from bone marrow into retina-specific cells.

In one embodiment of the invention, CCM is used to differentiate adult mesenchymal stem cells from bone marrow into retina-specific cells.

In a further embodiment of the invention, CCM is used to differentiate adult hematopoietic stem cells from bone marrow into retina-specific cells.

In a further embodiment of the invention, CCM is used to differentiate a mixture of adult mesenchymal and hematopoietic stem cells from bone marrow into retina-specific cells.

As stated above, the choroid-conditioned medium may, when used in the differentiating medium of the invention, comprise one or more factors selected from the group consisting of members of the FGF family FGF: “fibroblast growth factor”), members of the NT family (NT: “neurotrophin”), members of the BMP family (BMP: “bone morphogenic protein”), PDGF (“platelet-derived growth factor”), EGF (“epidermal growth factor”), BDNF (“brain-derived neurotrophic factor”), CNTF (“ciliary neurotrophic factor”), HGF (“hepatocyte growth factor”) and NGF (“nerve growth factor”) or subtypes thereof.

In preferred embodiments of the invention, the member of the FGF family is preferably basic fibroblast growth factor bFGF (FGF-2), the members of the neurotrophin family are preferably NT-3 and NT-4, and the member of the BMP family is preferably BMP-4.

The invention further relates to the use of retina extract (RE) for differentiating stem cells from bone marrow into retina-specific cells.

RE is preferably used according to the invention for differentiating adult mesenchymal stem cells and/or hematopoietic stem cells from bone marrow into retina-specific cells.

The invention also relates to the use of choroid-conditioned medium (CCM) and retina extract (RE) for differentiating stem cells from bone marrow into retina-specific cells.

TABLE 1
Pattern of expression of antigens in various
retina-specific cells of the invention

| Cell type | Positive signal | Negative signal |
|---|---|---|
| Retinal pigment | RPE65 | IRBP (“interphoto- |
| epithelium | | receptor-retinol- |
| | | binding protein”) |
| | ZO-1 | CD31 |
| | Occludin | CD34 |
| | CD36 | |
| | Cytokeratin 7, 8, 18, 19 | |
| | S-100 | |
| Photoreceptors | Rhodopsin (rods) | |
| | Calbindin (cones) | |
| | PKC (predominantly | PKC (cones) |
| | α isoform, only in | |
| | rods) | |
| | S-Antigen (S-Ag; | |
| | adult cells and in | |
| | later stages of | |
| | development | |
| Müller's cells | S-100 | PKC |
| (and astrocytes) | GFAP (“glial | |
| | fibrillary acidic | |
| | protein”) | |
| Amacrine cells | GABA (“gamma-amino- | |
| | butyric acid”) | |
| Gangliocytes | Neurofilament | |
| Bipolar cells | PKC | S-100 |
| Horizontal cells | Calbindin D | |
| Choroidal vascular | CD31 | |
| endothelium | | |

The invention further relates to retina-specific cells which are derived from stem cells and can be obtained by the method of the invention. A particular feature exhibited by these retina-specific cells of the invention which are in isolated form is a specific expression pattern (see Table 1) which is characterized by expression (cf. “Positive signal” column) or undetectable expression (cf. “Negative signal” column) of particular antigens located on the surface or intracellularly in the cytoplasm. Apart from the antigens RPE65 and rhodopsin, the investigated antigens are not specific for the cell type. However, since the investigated antigens are tissue-specific for retinal and also neural tissue, they make it possible, in addition to the morphological differences in the cells, to differentiate the retina-specific cells from the stem cells from which they are derived (see above) by immunostaining and interpretation of the characteristic staining results (positive/negative staining).

The retina-specific cells of the invention open up a wide field for genetic modification and therapy. In one embodiment of the invention there is transfection or transduction of the isolated stem cells from bone marrow per se or the retina-specific cells eventually differentiated therefrom with one or more genes. In a preferred embodiment of the invention there is transfection with one or more human retina-specific genes, as transgenes, of the isolated stem cells following the isolation from bone marrow or during the differentiation method following the expansion in step a) of the method, following the differentiation in step b) of the method, or of the retina-specific cells differentiated therefrom.

Retina-specific transgenes mean herein those genes which are naturally expressed in healthy retinal cells but not in the undifferentiated or differentiated stem cells.

Autologous stem cells and the retinal cells of a patient exhibit identical defects in their genomes which lead on activation of the relevant genes through the absence of or faulty expression to the establishment of a pathological state, e.g. in the retina in the case of retinal cells. Targeted gene therapy of such diseases, e.g. of retinitis pigmentosa, is possible by transfection of the stem cells used according to the invention or of the retina-specific cells differentiated therefrom before transplantation of the cells with a healthy copy of the defective gene. If genes are introduced into the stem cells during the method of the invention, they are preferably retained also in the retina-specific cells differentiated out of the stem cells and express the transfected gene after transplantation into the recipient also at the transplantation site. Methods for transfecting cells with transgenes are well known to the skilled worker [cf. SAMBROOK, J. et al. (2001) Molecular Cloning: A Laboratory Manual. Cold Spring Harbor Laboratory Press].

The gene constructs used for transfecting the stem cells or the retina-specific cells differentiated therefrom can have various designs and compositions known to the skilled worker.

Ideally, defective or missing genes ought to be repaired or replaced in their natural context, but this cannot in practice be achieved according to the current state of the art. It is therefore necessary for the missing or defective genes to be introduced initially into the genome of the stem or retina-specific cells and be expressed ectopically there. In order to ensure stable expression and transmission of the introduced genes to the daughter cells during cell division, the vectors which ought to be used according to the state of the art are retroviral [Baum et al., Curr Opin Mol. Ther. 1999 October; 1(5): 605-612] or lentiviral [Trono, Gene Ther 2000; 7: 20-33], and possibly also AAV vectors [Monahan & Samulski, Gene Ther 2000; 7: 24-30]. The viruses from which these vectors are derived are distinguished by being naturally integrated stably in the target cell genome and moreover being transmitted further like endogenous genes.

Following transduction with conventional vectors derived from γ retroviruses there would be uncontrolled expression of the introduced transgene. The level of this expression can, however, be determined within relatively wide limits beforehand through the choice of suitable viral promoters [Baum et al., Curr Opin Mol. Ther. 1999 October; 1(5): 605-612; Wahlers et al., Gene Ther. 2001 March; 8(6): 477-486]. On the other hand, the use of so-called SIN (“self-inactivating”) vectors allows the viral promoters to be replaced by any other promoter of choice [Kraunus et al., Gene Ther. 2004 November; 11(21): 1568-1578). For reasons of biosafety, SIN constructs are used exclusively with lentiviral (ordinarily HIV-derived) vectors. Since SIN vectors lack the viral promoter and enhancer elements, they might possibly be associated with a smaller risk of harmful side effects (“insertion mutagenesis”) [von Kalle et al., Stem Cell Clonality and Genotoxicity in Hematopoietic Cells Gene Activation Side Effects Should Be Avoidable. Seminars in Hematology, in press]. For the context given here, SIN vectors are of interest in particular because they allow the use of gene-specific [Moreau-Gaudry et al., Blood. 2001; 98: 2664-2672] or else regulatable or inducible promoters. The use of retina-specific promoters would certainly be the optimal solution. Inducible systems are currently in most cases based on the tetracycline system of Gossen & Bujard [Proc Natl Acad Sci USA. 1992; 89(12): 5547-51]. With such systems it is possible to suppress expression of the transgene during culturing by adding the respective inhibiting substance to the respective culture medium during the proliferation of the stem cells or the differentiation of the stem cells into retina-specific cells. If the patient does not receive this substance following transplantation of the retina-specific cells, the inhibition is terminated, the promoter is activated and the transgene is expressed.

Transfection with one or more foreign genes makes it possible on the one hand to introduce into the cells the genes which are necessary for maintenance of cell-typical metabolic activities in the retina-specific cells, but also included on the other hand is transfection of genes which confer novel functions on the retina-specific cells or label the cell. In a particularly preferred embodiment of the invention, the cells are transfected with the green fluorescent protein (GFP), the enhanced green fluorescent protein (eGFP) or the lacZ gene as marker or reporter gene for labeling the cells [cf. ALLAY, J. A. et al. (1997) Hum Gene Ther 8: 1417; AYUK, F. et al. (1999) Gen Ther 6: 1788-1792; FEHSE, B. et al. (1998) Gen Ther 5: 429-430].

The invention further relates to cell preparations which comprise retina-specific cells of the invention as isolated cells. Such cell preparations can be employed for storing or transporting the cells.

Cell preparations may comprise isolated vital retina-specific cells of the invention which are characterized by absent or undetectable expression of markers selected from the group consisting of IRBP and CD34 or by the expression of at least one of the markers selected from the group consisting of RPE65, ZO-1, occludin, CD36, cytokeratin 7, cytokeratin 8, cytokeratin 18, cytokeratin 19, S-100, rhodopsin (in rods), calbindin (in cones), PKC, S-antigen, GFAP, GABA and neurofilament, in an amount of at least 1, preferably 1-50%, in a particularly preferred manner from 50 to 70%, and in an extremely preferred manner from 70 to 90%, based on the total number of cells present in the preparation, in a suitable medium, with all integral values (i.e. 11, 12, 13, . . . 90%) being expressly included in the aforementioned range of values. Preference is given to cell suspensions in a cell-compatible cell culture or transport medium such as, for example, a standard medium selected from the group consisting of RPMI, medium 199, DMEM (low glucose; this medium corresponds to modified Eagle's medium (Gibco 31885) with or without HEPES as addition and Iscove's medium, in each case alone or as 1/1 mixture with Ham's F12 nutrient mixture. The medium may further be a special medium selected from the group consisting of medium human endothelial SFM (Gibco 11111), START V (Biochrom F8075) and Neurobasal or Neurobasal-A medium (Gibco 21103 or 10888) with or without Ham's F12 nutrient mixture as addition.

Also suitable are deep-frozen cell preparations in which the cells have been sedimented by centrifugation and taken up for example in 90% FCS and 10% DMSO. 10% methylcellulose or DMSO are added as adjuvant to the cryomedium in order to assist survival of the cells during the cryopreservation. In the case of serum-free treatment of the cells it is additionally necessary to add protective proteins to which the sensitive proteins can adhere and thus are protected during the cryopreservation. These are preferably added as albumin. It is also possible for the cells to be taken up in serum-free cryomedium (e.g. cryo-SFM (Promocell C-29910) instead of in differentiating medium. In this connection, cryomedia are media which allow the cells to be deep-frozen without damaging the cells.

In one embodiment of the invention following the differentiation, the differentiated retina-specific cells are separated from undifferentiated stem cells in order to achieve the maximum possible enrichment of retina-specific cells of the invention with simultaneous depletion of undifferentiated stem cells. Separation of the undifferentiated stem cells from the differentiated retina-specific cells is effected with the aid of (surface) antigens which are expressed specifically on the (partly) differentiated retina-specific cells but not, or undetectably, on the undifferentiated stem cells. Antigens which can be used for such a separation are for example CD36 or S-100, and all antigens from the “Positive signal” column (see Table 1). Separation of the cells very substantially prevents quantitatively large amounts of cells capable of differentiation being present besides the retina-specific cells with a purely proliferative capacity in the mass of cells. It is additionally possible to ensure in this way that the cell counts adjusted for example during the production of a pharmaceutical composition in fact represent the retina-specific cells of the invention.

In a further preferred embodiment of the invention, the differentiation is followed not by separation of the differentiated retina-specific cells from undifferentiated stem cells but by enrichment of the differentiated cells or depletion of the undifferentiated stem cells. Such an enrichment or depletion is likewise effected with the aid of specific surface antigens.

Examples of methods known in the art which can be used for sorting particular surface marker cells include immuno magnetic bead sorting (cf. ROMANI, et al. (1996) J Immunol Methods 196: 137-151], fluorescence-activated cell sorting (FACS) and magnetic-activated cell sorting (MACS) [loc. cit.]. Further methods of these types are known to the skilled worker.

In one embodiment of the invention, the retina-specific cells of the invention are employed per se for producing a pharmaceutical composition for the treatment of diseases which are associated with acquired or congenital dysfunction of the cells of the retinal pigment epithelium, of the cells of the adjacent structures of the whole retina and of the choroid, and of further tissues of the eye, or for regenerating the optic nerve (nervus opticus), e.g. in the event of or following glaucomatous damage.

The bone marrow from which the stem cells are isolated may be of autologous or allogeneic origin. The term “autologous” refers to tissues or cells which have been taken from the same individual who is to receive the differentiated retina-specific cells as transplant. An allogeneic origin indicates that the bone marrow donor and the recipient of retina-specific cells which have been differentiated out of the bone marrow are different, but belong to the same species, i.e. donor and recipient are human.

In a particularly preferred embodiment of the invention, the retina-specific cells are autologous cells, i.e. the stem cells from the bone marrow originate from the patient who is to be treated with the retina-specific cells differentiated out of these stem cells. In such a case, giving the retina-specific cells differentiated out of stem cells does not cause any immunological problems in the form of cell rejection because the cells and the recipient have identical tissue types.

The pharmaceutical products may comprise the retina-specific cells of the invention, i.e. partly and/or completely differentiated cells, suspended in a physiologically tolerated medium. Examples of suitable media are standard media selected from the group consisting of RPMI, medium 199, DMEM (low glucose; this medium corresponds to modified Eagle's medium (Gibco 31885) with or without HEPES as addition and Iscove's medium, in each case alone or as 1:1 mixture with Ham's F12 nutrient mixture. The medium may further be a special medium selected from the group consisting of medium human endothelial SFM, START V and Neurobasal or Neurobasal-A medium with or without Ham's F12 nutrient. On use of the special media for producing a pharmaceutical composition, care must be taken that the media are suitable for this use and comprise no hormones, peptides or the like to which the patient might be sensitive. Care must absolutely be taken to ensure that the medium used for transplantation contains no serum. Substitutes which can be employed are physiological solutions, e.g. Ringer's solution.

The retina-specific cells of the invention which are characterized by at least one of the markers selected from the group consisting of RPE65, ZO-1, occludin, CD36, cytokeratin 7, cytokeratin 8, cytokeratin 18, cytokeratin 19, S-100, rhodopsin (in rods), calbindin (in cones), PKC, S-antigen, GFAP, GABA and neurofilament are preferably present in such pharmaceutical compositions in an amount of at least 50%, preferably at least 60%, based on the total number of cells present in the product, with all integral values (i.e. 51, 52 . . . 59 and 61, 62 . . . 99, 100) being expressly included in the aforementioned range of values. The pharmaceutical products may optionally comprise further pharmaceutically acceptable excipients and/or carriers.

In a further preferred embodiment of the invention, at least 1×10⁴ retina-specific cells of the invention are present per μl of the pharmaceutical products. However, preferably not more than 5×10⁴ retina-specific cells of the invention are present per μl in order to avoid agglomeration of the cells.

Preferred administration forms for the in vitro differentiated retina-specific cells are injection, infusion or implantation of the cells into a specific assemblage of cells in the eye in order to achieve adhesion of the cells there on one hand through direct contact with the assemblage of cells, and undertaking functions of the damaged tissue through differentiation appropriate for the tissue.

A particularly preferred administration form is injection of the in vitro differentiated retina-specific cells. This is preferably effected by local intraocular implantation.

The local intraocular administration particularly preferably takes place into the retina (intraretinal, cf. GUO, Y. et al. (2003) Invest Opthalmol V is Sci 44(7): 3194-3201), underneath the retina (subretinal, cf. WOJCIECHOWSKI, A. B. et al. (2002) Exp Eye Res 75(1): 23-37) or into the vitreous near the retina (intravitreal, cf. JORDAN, J. F. et al. (2002) Graefe's Arch Clin Exp Opthalmol 240(5): 403-407).

A further preferred embodiment of the invention relates to the systemic infusion of the in vitro differentiated retina-specific cells of the invention via the bloodstream so that the cells accumulate in the retina.

Preferred examples of indications relevant in this connection are retinitis pigmentosa, age-related macular degeneration or glaucoma. The term “glaucoma” refers in this connection to a number of degenerations of the nerve fibers and of the optic nerves which are ascribed to an in most cases abnormal intraocular pressure. This is characterized by loss of gangliocytes of the retina and of the nerve fibers, and atrophy of the optic nerve. The term “glaucoma” encompasses according to the invention all types of glaucomas, i.e. high-, normal-, low-pressure glaucoma, open-angle glaucoma, PEX glaucoma etc.

The treatment according to the invention of glaucoma includes the replacement of destroyed gangliocytes and nerve cells in the retina and in the optic nerve by giving stem cells from bone marrow which have been partly or completely differentiated according to the invention into retina-specific cells to form a replacement for the destroyed cells.

The retina-specific cells differentiated according to the invention from hematopoietic stem cells can also be used to treat the damage to the choroid associated with diabetes (diabetic retinopathy). Administration of the cells of the invention stabilizes the vessels which have become fragile as a consequence of the diabetes, and thus reduces or prevents the occurrence of retinal hemorrhages.

Consequently, preferred embodiments of the invention are the use of the retina-specific cells for producing pharmaceutical compositions for the treatment of retinitis pigmentosa, age-related macular degeneration or glaucoma.

It is further preferred according to the invention to use retina-specific cells partly or completely differentiated from hematopoietic stem cells for producing a pharmaceutical composition for the treatment of disorders which are characterized by a degeneration of the vascular structures of the choroid, e.g. of diabetic retinopathy in diabetes.

The cells may be, as described above, of autologous or allogeneic origin, i.e. the bone marrow from which the mesenchymal or hematopoietic stem cells have been isolated originates from the body of the recipient or of a representative of his species.

The invention is explained and described below by means of examples without being restricted to these exemplary embodiments:

### EXAMPLE 1

### Isolation of Bone Marrow Cells

Adult mesenchymal stem cells were obtained from bone marrow samples (aspirates) which were taken from a live donor during a minor operation. The stem cells were separated out of the sample by centrifugation on a Ficoll gradient (Biochrom K G, “Biocoll Separation Solution, isotonic solution; density 1.077 g/ml). The cells from the mononuclear cell layer were resuspended in a culture medium (DMEM, low glucose) supplemented with 10% fetal calf serum (FCS) and cultured in uncoated tissue culture-treated plastic culture dishes (polystyrene).

The first culturing after isolation of the cells generally takes place in 24-well plates. Depending on the size of the culture, also suitable are 12-well plates, 6-well plates, T25 culture bottles or T75 culture bottles.

The cell cultures obtained by this method can be cultured in DMEM (low glucose) medium with 10% FCS for several months by passaging them every 7 to 14 days depending on the seeding density, the influence of the donor, the age of the culture and when subconfluence (60-80%) is reached (see Example 2).

### EXAMPLE 2

### Passaging of the Mesenchymal Stem Cells

The mesenchymal stem cells were passaged by removing the culture medium and non-adherent cells from the growing mesenchymal stem cells adhering to the plastic by aspiration or lifting off. The adherently growing mesenchymal stem cells which adhered to the culture dish were washed 1-2 times with PBS (which must contain no calcium or magnesium ions) in order to remove further non-adherent cells. This was followed by incubation at room temperature in a trypsin/EDTA solution (trypsin 0.02%, EDTA 0.05% in calcium or magnesium ion-free PBS) for 1 minute. After completion of the incubation, the trypsin/EDTA solution was aspirated off again and the cells were left at room temperature for a further 2-3 minutes. The culture vessel was then cautiously shaken by manual tapping in order to detach the cells from the surface of the culture vessel by the mechanical stress. The detached cells were suspended in DMEM low glucose/10% FCS.

The cell count was determined either by mixing 10 μl of the suspension with 10 μl of Trypan blue solution, pipetting into a Neubauer chamber and counting dead (stained cells) and vital (unstained) cells under the microscope, or diluting 0.5 ml of the suspension with 12.5 to 19.5 ml of an isotonic saline solution (specifically for use in a Coulter counter cell counter) and counting the latter in a Coulter counter cell counter.

The total number of vital cells is in both cases calculated taking the dilution into account. The cells were then passaged by seeding in to appropriate uncoated culture vessels and culturing further in the same culture medium as used for seeding. It may be necessary for this purpose to dilute the cell suspension further with culture medium.

The cell suspension can further be sedimented by centrifugation, the sedimented cells be suspended in freezing medium (90% FCS+10% DMSO) and be cryopreserved in liquid nitrogen.

### EXAMPLE 3

### Production of Retina-Specific Cells by Using CCM

Firstly 4 to 8 ml of choroid-conditioned medium (CCM) were generated for differentiating adherently growing mesenchymal stem cells after isolation from bone marrow (see Example 1 and 2). Two eyes (corresponding to a pair of eyes) from an allogenate donor were treated as follows to produce 4 ml of CCM:

Firstly, the anterior segment, the vitreous and the neurosensory retina of the eye were removed, followed by dissection of the choroids and/or fragments thereof with scissors and forceps [cf. VALTINK, M. et al. (1999) Graefe's Arch Clin Exp Opthalmol 237: 1001-1006; VALTINK, M. & ENGELMANN, K. (2002) In: WILHELM, F., DUNCKER, G. I. W., BREDEHORN, T. (editors) Augenbanken. Walter de Gruyter Verlag Berlin New York, pp. 75-87). The choroid is usually still complete. Blood, loosely adherent dead cells and tissue fragment which would interfere with further treatment of the choroid were removed by washing with 2 ml of phosphate-buffered saline (PBS) per choroid. This was followed by incubation in a collagenase solution (1:1 collagenase IA and IV [cf. VALTINK, M. et al. (1999) Graefe's Arch Clin Exp Opthalmol 237: 1001-1006; VALTINK, M. & ENGELMANN, K. (2002) In: WILHELM, F., DUNCKER, G. I. W., BREDEHORN, T. (editors) Augenbanken. Walter de Gruyter Verlag Berlin New York, pp. 75-87); final concentration 0.5 mg/ml; 2 ml of solution per choroid) in an incubator under 5% CO₂ at 37° C. for about 4 to 16 hours in order to release cells of the retinal pigment epithelium from the choroidal tissue. An incubation time of 1 to 4 hours is sufficient on use of higher final concentrations of collagenase (e.g. 1 mg/ml). In some cases the choroid loses cohesion through the enzymic activity of collagenase and disintegrates on transfer into new medium.

The subsequent conditioning process is not impaired thereby. The enzymic activity was subsequently stopped by adding an excess of serum-containing culture medium (DMEM+FCS, see Example 1). The choroidal tissue was then transferred into 2 ml of culture medium consisting of F99 medium supplemented with 1% FCS per choroid, i.e. 4 ml of medium per pair of eyes. The tissue was incubated in an incubator under 5% CO₂ at 37° C. for 4 days.

The enzymic activity of the collagenase is only partly stopped by adding an excess of serum-containing culture medium because commercial collagenases exhibit, besides the proteolytic cleavage of collagens, as main activity further proteolytic activities which are difficult to inactivate and which are directed against other protein structures. This non-inactivatable residual activity is, however, small and has no influence on the formation of the conditioned medium and its use for cell culturing and differentiation.

CCM formed as supernatant during this incubation. The latter was separated from the choroidal tissue by centrifugation at room temperature and at 300×g for 10 min. The resulting supernatant was used directly as addition for differentiation or was deep-frozen at −20° C.

About 15 000 stem cells derived from bone marrow (see Example 1 and Example 2 for mesenchymal stem cells) and cultured for not more than 6 passages (see Example 2) were incubated with 5 ml of medium F99 (this is a 1:1 mixture of medium 199 and Ham's F12 nutrient mixture) which is supplemented with 1 to 10% of FCS, 1 μg/ml insulin, 1 mmol/l sodium pyruvate and 10% CCM in a T25 culture bottle for 14 to 21 days. The differentiating medium was changed 2 to 3 times a week, thus resulting in a total amount of about 30 to 45 ml of differentiating medium required to differentiate a donor culture.

After about 5 days, a decline in the rate of division and distinct morphological changes tending towards a stellar morphology was observed in the cells (cf. FIG. 2). In addition, an accumulation of dark granules around the core was to be observed.

After 10 to 14 days in culture, the cells began to develop a neuronal morphology, with dendritic, frequently branched offshoots, often accompanied by formation of podia at points of contact with adjacent cells (cf. FIG. 3b and FIG. 4).

After 19 days in culture, the cells were further characterized by adding, after removal of the culture medium, to unfixed cells and to cells previously fixed with 5% strength formalin at 4° C., a solution of the amino acid derivative L-3,4-dihydroxyphenylalanine (L-DOPA, 0.1% strength solution in PBS with neutral pH, equivalent to 1 mg/l) to detect active tyrosinase, the key enzyme of melanogenesis, and incubating at 37° C. for 45 min. After completion of the incubation for 45 minutes, the solution was renewed in each case until the total duration of the incubation reached 3 hours, attention being paid every 30 min to the progress of the reaction.

It is checked with the aid of this detection whether the stem cells are also able to differentiate into pigmented cell types such as, for example, cells of the retinal pigment epithelium or melanocytes. The capability of the cells for pigmentation via the tyrosinase pathway is the crucial differentiation criterion in this case. The detection is positive if blackish-brown particles consisting of melanin become visible as deposits formed from the added L-DOPA by the tyrosinase enzyme present in the cells and its derivatives, and the subsequent enzymes in this reaction chain.

Cells of this type were detectable after 19 days in culture.

### EXAMPLE 4

### Production of Retina-Specific Cells by Use of RE

Retina extract (RE) which was produced by homogenizing retinas was used to differentiate the adherently growing mesenchymal stem cells isolated from bone marrow (see Examples 1 and 2) and non-adherently growing hematopoietic stem cells (see Examples 1 and XY).

RE was produced as follows:

The neurosensory retina was dissected out of 10 human donor eyes. For this purpose, firstly the anterior segment and then the vitreous was removed from the eyes. The neurosensory retina of the eyes was then lifted using forceps and cut off with scissors at the optic disk. The resulting retinas were made up as a whole to a volume of 50 ml in a vessel with PBS and homogenized with addition of proteinase inhibitors (e.g. 1 tablet of complete protease inhibitor cocktail (Roche) per 50 ml of homogenate) in a manual or tissue homogenizer made of glass on ice. The supernatant, which represents the RE, was obtained by centrifugation at 500×g for 15 min and further centrifugation at 10 000×g for 45 min. The RE was then sterilized by filtration through a 0.22 μm sterilizing filter.

Differentiation of the passaged stem cells with RE-containing differentiating medium took place in analogy to Example 3. However, a difference was that 1% RE was added instead of the CCM to the differentiating medium. The differentiation of the stem cells into retina-specific cells took place during culturing in the differentiating medium for 2 to 3 weeks.

### EXAMPLE 5

### Analysis of the Composition of the Choroid-Conditioned Medium by MALDI-TOFF

Although it was possible to show the effect of CCM on the proliferation and differentiation of the mesenchymal stem cells (MSC) and the cells of the retinal pigment epithelium (RPE) (see Examples 3 and 4), the exact composition of this medium was initially unknown.

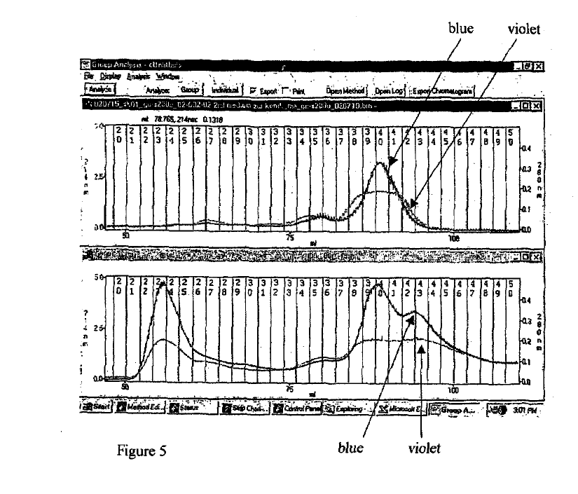

In order to establish the composition of the CCM, the supernatant from the choroid culture from Example 3 was fractionated into 80 fractions by gel filtration on a Superdex® column (Pharmacia Biotech). A chromatogram of the individual fractions was constructed by determining the protein content of the individual fractions in a chromatograph by measuring the absorption at 214 and 280 nm (see FIG. 5).

On the basis of the signal peaks visible in the chromatogram, the fractions in each case assignable to a group of peptide/proteins were combined. For example, in each case fractions 22-37, fractions 38-41 and fractions 42-46 were combined separately. Fractions 22-37 contain smaller peptide/protein molecules which were not present in this concentration before conditioning of the medium and are newly synthesized smaller peptides/proteins or degradation products of serum. Fractions 38-41 contain peptides/proteins from the largest peak which was present in the medium before the conditioning, but the amount thereof was increased through the incubation with the choroid.

This indicates that not all the serum proteins originally added to the medium had been consumed by the conditioning. Fractions 42-46 contain peptides/proteins which were not present in the medium before the conditioning. The size of the peptides/proteins present in this peak suggest that they are not degradation products of serum but must originate from the choroid and have been released into the medium during conditioning thereof.

After the fractions were combined they were tested for their biological activity by adding them to a culture medium used for culturing human mesenchymal stem cells and human retinal pigment epithelial cells.

For this purpose, normal human RPE cells from two donors from the first and third passage were seeded with a seeding density of 500 cells per well in 12-well culture dishes with F99 medium which was supplemented with 10% FCS, and incubated overnight so that the cells were able to adhere to the dish. The medium was then replaced by the test media which were composed of F99, 5% FCS and the fractions from the fractionation of the CCM. F99 mixed with 5% FCS without addition of a CCM fraction was employed as negative control, and F99 mixed with 5% FCS and CCM was employed as positive control. On the one hand, CCM without fractionation as a whole and, on the other hand, also after fractionation and renewed combining was employed as positive control. In each case, 3 wells were provided with the same medium, so that each fraction was tested twice with three replicates in each case. After 12 days for test 1 and 14 days for test 2, the cells were detached from the culture plate by trypsinization, and the cell count in the individual wells was established by counting.

The biological activity of the fractions was determined from the difference of the cell count at the end and at the start of the culturing. The difference found in this way corresponds to the cells produced by proliferation during the culturing, which changes as a function of the biological activity of the added CCM fraction. In this case, biologically active fractions increase the proliferation of the cells compared with the negative control, although the maximum increase reaches the value of the positive control.

The test with the human mesenchymal stem cells was carried out analogously, but a difference was that 5000 cells were seeded per well in 24-well culture dishes.

Fractions with a biological activity which had a positive effect on the proliferation of the stem cells were subsequently subjected to analysis by MALDI-TOF mass spectrometry in order to identify the peptides or proteins in the fraction which were the basis for the biological activity of the fractions. For this purpose, the proteins of the corresponding fraction were first fractionated by 2D gel electrophoresis in a protein gel, and the proteins in the excised protein band were proteolytically restricted. The resulting peptides were extracted from the gel and were then characterized by mass spectrometry and identified on the basis of their physical data through a database search.

1-38. (canceled)

39. A method for differentiating bone marrow stem cells into retina-specific cells comprising culturing said bone marrow stem cells in a differentiating medium comprising a composition selected from the group consisting of choroid-conditioned medium, retina extract, and combinations thereof.

40. The method of claim 39, further comprising:
expanding said bone marrow stem cells in a suitable culture medium prior to said culturing said bone marrow stem cells in said differentiating medium, and then
isolating said retina-specific cells from said differentiating medium following said culturing said bone marrow stem cells in said differentiating medium.

41. The method of claim 39, wherein said bone marrow stem cells include mesenchymal stem cells.

42. The method of claim 41, wherein said mesenchymal stem cells express at least two antigens selected from the group consisting of CD59, CD90, CD105 and MHC I.

43. The method of claim 41, wherein said bone marrow stem cells include adherent mesenchymal stem cells.

44. The method of claim 39, wherein said bone marrow stem cells include hematopoietic stem cells.

45. The method of claim 44, wherein said hematopoietic stem cells express at least one antigen selected from the group consisting of CD34 and CD45.

46. The method of claim 39, wherein said bone marrow stem cells include mesenchymal and hematopoietic stem cells.

47. The method of claim 39, wherein said differentiating medium comprises from 1 to 20% choroid-conditioned medium or from 0.1 to 5.0% retina extract.

48. The method of claim 39, further comprising:
isolating said retina-specific cells from said differentiating medium following said culturing said bone marrow stem cells in said differentiating medium, and then
suspending said retina-specific cells in a liquid culture medium.

49. The method of claim 48, wherein said liquid culture medium is selected from the group consisting of
RPMI;
DMEM;
Iscove's medium;
medium 199;
human endothelial-SFM;
START V;
Neurobasal/Neurobasal-A medium and their supplements N-2 or B27; and
a 1:1 mixture of Ham's F12 nutrient mixture and a medium selected from the group consisting of RPMI, DMEM, Iscove's medium and medium 199.

50. The method of claim 48, further comprising:
adding a cryoprotectant and a protein to said liquid culture medium after said suspending said retina-specific cells, and then
deep-freezing said retina-specific cells.

51. The method of claim 50, wherein said cryoprotectant is selected from the group consisting of DMSO and methylcellulose, and said protein is selected from the group consisting of serum and albumin.

52. The method of claim 39, wherein said culturing of said bone marrow stem cells is for a time period ranging from 3 to 21 days.

53. The method of claim 39, wherein said culturing of said bone marrow stem cells is for a time period of 1 to 28 days in retina extract-containing differentiating medium following a time period of 3 to 14 days in a choroid-conditioned medium-containing differentiating medium.

54. The method of claim 39, wherein said culturing of said bone marrow stem cells is in a special medium for neuron cultures following a time period in a choroid-conditioned medium-containing differentiating medium.

55. The method of claim 54, wherein said special medium for neuron cultures is Neurobasal or START V.

56. The method of claim 39, wherein said culturing of said bone marrow stern cells creates a collection of bone marrow stem cells with a density ranging from 0.5×10³ to 2.5×10³ cells per cm².

57. The method of claim 39, wherein said culturing of said bone marrow stem cells, cells are seeded with a density ranging from 2×10² to 5×10³ cells per well in a 24-well plate.

58. The method of claim 39, further comprising transfecting one or more foreign gene into said bone marrow stern cells at a time selected form the group consisting of following isolating said bone marrow stem cells from said bone marrow, during an expanding of said bone marrow stem cells, following said culturing of said bone marrow stem cells, and following an isolating of said retina-specific cells.

59. The method of claim 39, wherein said choroid-conditioned medium comprises one or more growth factors selected from the group consisting of a member of the fibroblast growth factor family, a member of the neurotrophin family, a member of the bone morphogenic protein family, a platelet-derived growth factor, an epidermal growth factor, a brain-derived neurotrophic factor, a ciliary neurotrophic factor, a hepatocyte growth factor and a nerve growth factor.

60. The method of claim 59, wherein said member of said fibroblast growth factor family is a basic fibroblast growth factor selected from the group consisting of basic fibroblast growth factor and fibroblast growth factor-2.

61. The method of claim 59, wherein said member of said neurotrophin family is selected from the group consisting of neurotrophin-3 and neurotrophin-4.

62. The method of claim 59, wherein said member of said bone morphogenic protein family is bone morphogenic protein-4.

63. A mixture comprising:
(a) one or more bone marrow stem cells, and
(b) a differentiating medium comprising a composition selected from the group consisting of choroid-conditioned medium, retina-extract medium, and combinations thereof.
